(19) United States
(12) Patent Application Publication
(10) Pub. No.: US 2025/0034552 A1
(43) Pub. Date: Jan. 30, 2025

(54) COMPOSITIONS AND METHODS FOR ISOLATION OF RIBOSOME PROTECTED MESSENGER RNA (mRNA) FOOTPRINTS OR FRAGMENTS AND KITS THEREOF

(71) Applicant: Prashant Karbhari Khade, Carlsbad, CA (US)

(72) Inventor: Prashant Karbhari Khade, Carlsbad, CA (US)

(21) Appl. No.: 18/781,722

(22) Filed: Jul. 23, 2024

Related U.S. Application Data
(60) Provisional application No. 63/528,751, filed on Jul. 25, 2023.

(51) Int. Cl. C12N 15/10

(52) U.S. Cl. CPC C12N 15/1062; C12N 15/1017

(57) ABSTRACT

The invention provides components, compositions and methods for isolation and purification of ribosome protected mRNA footprints or fragments (RPFs) and kits thereof. The method of the present invention uses protein concentrator column having certain molecular weight cut-off membrane for removing contaminant, concentrating monosomes, and isolating ribosome protected mRNA footprints. This invention is suitable to any cell types and organism for purification of ribosome protected mRNA footprints. This method reduces steps, instrumentation and cost involved in purification of ribosome protected mRNA footprints from any cells.

### CROSS-REFERENCE TO RELATED APPLICATIONS

This non-provisional application claims benefit of provisional application U.S. Ser. No. 63/528,751 filed Jul. 25, 2023, the entirety of which is hereby incorporated by reference.

### FEDERAL FUNDING LEGEND

This invention was made with government support under the Small Business Innovation Research (SBIR) grant 1R43GM142408-01 A1 awarded by the National Institute of Health (NIH). The government has certain rights in the invention.

### FIELD OF THE INVENTION

The present invention relates generally to the field of molecular biology. More particularly it is related to the fields of translating mRNA specially it concerns a quick and easy isolation of ribosome protected mRNA footprints from any cell or biological samples. The method is particularly suitable for isolating and preparing ribosome footprints for next generation sequencing applications.

### BACKGROUND OF THE INVENTION

During mRNA translation, ribosome translate mRNA sequence into respective protein sequence with high precision and accuracy. Recent experimental evidence demonstrate that many different proteins can be produced from single mRNA molecule and their rate of translation can vary both time and space in the cell. Cells can adapt to external and internal stimuli or signals by precisely modulating the mRNA translational profile. Therefore, mRNA translation transient processes are known to alter the cellular function and their fate by producing specific proteins and peptides. All these claims put forward the idea of determining mRNA coding frames and annotate the new coding sequences of genomes before making any conclusion about the mechanism and understanding of the disease state (cellular pathways) solely based on mRNA expression. Hence, it is very important to determine in vivo mRNA translation at sub-codon resolution to understand its correlation to cellular state.

At present there are three technologies that can detect mRNA translation at a sub-codon resolution: mRNA open reading frame prediction by bioinformatics, peptide sequence determination by mass spectrometry and identification of ribosome locations on mRNA by ribosome profiling. Bioinformatic prediction is a data-driven approach has limited applicability without experimental support. In mass spectrometry smaller length of polypeptide and their low concentration limits their detection. However, monitoring ribosome locations on mRNA by ribosome profiling is highly pursued and sensitive because it is based on next generation sequencing platform.

Methods to determine the ribosome locations or occupancy on mRNA is known for many decades reported first by Steitz in 1969. It involved treating initiating ribosomes by ribonuclease and isolating ribosome protected mRNA fragments to identify translation initiation site. Ribonucleases are known to degrade single standard RNA and leaving 25-30 nt length ribosome protected mRNA fragments. In 1988 Wolin and Peter Walter extended this approach for determining the distribution of translating ribosomes on specific mRNA. Ingolia and Weismann extended this approach to determine the global mRNA translation profile by tossing the term “ribosome profiling” in 2009. It involved isolation and purification of ribosome protected mRNA footprints and their alignment to the genome to understand global mRNA translation at sub-codon resolution.

Since then, ribosome profiling (RP) has been exploited by many researchers across the world to identify translation initiation, ribosome pausing and mRNA reading frames in many cellular conditions and diseases states of various organisms. Ribosome profiling consist of six steps: ribosome stabilization, cell lysate preparation, nuclease protection, isolation of ribosome protected fragments or footprints (RPFs), next generation library preparation for sequencing and RPF alignment to the genome. Though much work has gone into optimizing the RP since last 15 years, majority of modifications in the protocol has focused on nuclease digestion of stalled ribosome and in library preparation. Steps such as isolation and purification of ribosome protected fragments (RPFs) that contribute a large portion to the time and quality of the RPFs have been largely neglected. Currently, purification and isolation of RPFs involve purifying monosomes using sucrose gradient or cushion and then isolation of RPFs using urea-polyacrylamide gel. These purification and isolation steps are time consuming and require expensive ultracentrifuge and polysome fractionator instruments. Even after several modifications in the original RP technology, it still needs expertise and takes 4-5 days of intensive hands-on work. Given the power and potential for widespread use, there is an urgent and unmet need to modify present isolation of ribosome protected fragments or footprints (RPFs) for example; platform like RNA library preparation for sequencing has significantly improved over the years, becoming faster, artifact-free, and more cost-effective.

Additionally, all present footprint isolation methods require rRNA depletion which are present in the significant amount (60-90%) in the isolated RPFs. Further, rRNA depletion steps to remove rRNA create an artifact due to the non-specificity of rRNA depletion steps. For example, the presence of rRNA fragments in the enriched RPFs can increase background in the sample resulting in variation in enriched RPFs and hindering the detection of less abundant translating RPFs. Hence, one of the objects of the present invention is to provide a method suitable for enriching RPFs by reducing rRNA fragments which avoids one drawback of the prior art methods.

Hence, for widespread usage of RP we need a technology which can quickly isolate ribosome protected footprints (RPFs) with reduced instrumentation, cost, and expertise. Presently available kit has several limitation and artifacts. The present invention provides a method for fast and economical isolation of RPFs from any cell. Isolated RPFs by present invention is compatible with various small RNA library preparation kits for next generation sequencing and can be customized to meet the specific investigational need of different customers bases.

### SUMMARY OF THE INVENTION

It is an object of the present invention is to provide process or methods, compositions, and kit thereof which concerns a quick and easy isolation of ribosome protected mRNA fragments or footprints (RPFs) from any cell or tissue.

The present invention is directed to a method for isolation and purification of ribosome protected mRNA fragments or footprints. The method comprising the steps of (i) providing a suspension comprising monosomes; (ii) treating the suspension with a dissociation buffer comprising reagents that dissociate monosomes to release the ribosome protected mRNA fragments or footprints; and (iii) filtering the mixture of step (ii) through an ultrafiltration unit comprising molecular weight cut-off (MWCO) membrane under conditions to remove impurities and elute ribosome protected mRNA fragments through the column; wherein the ribosome protected mRNA fragments has a length of 25-35 nucleotides.

The present invention is directed further to a method for isolation and purification of ribosome protected mRNA fragments or footprints. The method comprising the steps of (i) loading a suspension comprising monosomes on an ultrafiltration column comprising a molecular weight cut-off (MWCO) membrane; (ii) adding about 20-30 fold of dilution buffer comprising reagents that reduce non-specific RNA-RNA interactions and inhibit RNase activity; (iii) filtering through the ultrafiltration column under conditions to remove impurities and obtain monosomes that is substantially free of impurities in a retentate solution; (iv) treating the retentate solution with a dissociation buffer comprising reagents that dissociate monosomes to release the ribosome protected mRNA fragments or footprints; and (v) filtering the above through the ultrafiltration column under conditions to remove impurities and elute ribosome protected mRNA fragments through the column; where the membrane in ultrafiltration unit has a molecular weight cut-off (MWCO) of about 50K to about 100K Daltons and where the ribosome protected mRNA fragments has a length of 25-35 nucleotides.

The present invention is directed further to a method for isolation and purification of ribosome protected mRNA fragments or footprints from a total cell lysate. The method comprising the steps of (i) loading the total cell lysate on an ultrafiltration column comprising a molecular weight cut-off (MWCO) membrane; (ii) treating with a medium comprising nucleases; (iii) adding about 20-30 fold of dilution buffer comprising reagents that reduce non-specific RNA-RNA interactions and inhibit RNase activity; (iv) filtering through the ultrafiltration column under conditions to remove impurities and obtain monosomes that is substantially free of impurities in a retentate solution; (v) treating the retentate solution with a dissociation buffer comprising reagents that dissociate monosomes to release the ribosome protected mRNA fragments or footprints; and (vi) filtering the mixture of step (v) through the ultrafiltration column under conditions to remove impurities and elute ribosome protected mRNA fragments through the column; where the total cell lysate comprises polysomes; where the membrane in ultrafiltration unit has a molecular weight cut-off (MWCO) of about 50K to about 100K Daltons, and where the ribosome protected mRNA fragments has a length of 25-35 nucleotides.

The present invention is directed further still to a method for small RNA library preparation. The method comprising (i) obtaining purified ribosome protected mRNA fragments or footprints of the invention and treating the ribosome protected mRNA fragments with a 5′ and 3′ end repair buffer to promote 5′ phosphorylation and 3′ dephosphorylation.

In one embodiment, cell lysis buffer is comprised of specific composition that efficiently lyse cells, stabilize ribosome conformation and maintain high nucleases activity to generate uniform nucleotide length ribosome protected mRNA footprints. Ribonucleases are known to degrade single standard RNA leaving ribosome protected mRNA fragments. This process is also called “Ribonuclease protection reaction” and is performed under conditions that optimize RNA degradation reaction step wherein uniform nucleotide length of RPFs are obtained. However, molecular size of ribosomes varies with species from bacteria to eukaryotes therefore they protect various lengths of mRNA. Additionally, the length of RPFs is dependent on ribosome functional states and ribonuclease protection conditions.

In another embodiment DNase-I (RNase-free) was added into ribonuclease protection reaction. DNase-I is enzyme known to degrade single and double stranded DNA from the sample. This step help to reduce the viscosity of total lysate by digesting chromatin in addition to removing contaminating DNA fragments and prepare sample for ultrafiltration step.

In one embodiment monosomes are purified and concentrated directly from nuclease treated whole cell lysate. This embodiment is based on purification and concentration of monosomes by removing unwanted impurities smaller than certain molecular weight cut-off (MWCO) column while retaining monosome fraction due to ribosome size ˜2.3-4.5 MDa depending on the species. All monosome free (proteins, DNA and RNA) contaminants smaller than MWCO are removed by repetitive dilution of nuclease treated total cell lysate followed by centrifugal filtration. In each dilution step concentration of contaminants is reduced and retained monosome purity is improved. This invention enhances the purity of RPFs by removing fragmented RNA and DNA contaminant, thus increasing useful reads in next generation sequencing library. The described invention herein avoids ribosome purification by affinity tag thus, any biases arise due to the heterogeneous population of ribosomes due to composition of ribosomal proteins or post-transcriptional ribosomal RNA modification or post-translation modification of ribosomal proteins in ribosomes.

In another embodiment retentate monosomes are dissociated by adding ribosome dissociation buffer which concomitantly release ribosome protected mRNA footprints and ribosomal subunits (small and large subunits) into the dissociation buffer. Released RPFs are isolated as a filtrate by centrifugal filtration having anything higher than 30 kDa MWCO membrane. 25-35 nucleotide length RPFs can easily passed through >30 kDa MWCO membrane due to small size than MWCO. Dissociated ribosome subunits are retained in the column due to their higher molecular weights 2.0-4.5 mDa.

In other embodiment, monosomes are also dissociated using puromycin with high salt treatment which is known to dissociate only elongating ribosomes which can be advantageous when isolating only translating RPFs. Additionally, monosomes are dissociated into its subunits using EDTA treatment. EDTA chelated magnesium ions involved in the stabilization of RNA structure.

In another embodiment RPFs are end repaired to make them compatible to various RNA library preparation kit for next generation sequencing. RPFs end repaired by treating with T4 PNK in the presence or absence of ATP to generate 52 phosphate and 32 hydroxyl ends. In the absence of ATP T4 PNK stoichiometrically transfers 3′-phosphates to itself. However, the necessity of this end repair step depends on the specific type of next-generation small RNA library preparation kit used.

In another embodiment DNase-I (RNase-free) was added into RPFs end repair reaction to remove any remaining contaminating DNA present in the isolated RPFs sample.

Another aspect of the present invention relates to a kit for isolation and purification of RPFs from cells-comprising biological samples. A kit comprising, (a) lysis buffer with specific composition important for efficient cell lysis and ribosome stabilization; (b) dilution or wash buffer with specific composition important for reducing non-specific RNA-RNA interactions and inhibiting RNase activity; (c) dissociation buffer with specific composition to dissociate monosomes to release RPFs; (d) 52 and 32 end repair buffer has specific composition for 52 phosphorylation and 32 dephosphorylation of RPFs which further maintain the activity of DNase-I enzyme for DNA degradation; (e) certain molecular weight cut-off (MWCO) columns for monosome purification and RPFs isolation; and (f) instructions or steps for isolation of RPFs.

In present invention all steps can be performed in one column which enables isolation of RPFs by means of automation on equipment's or instruments. Many previously disclosed methods do not lend themselves to automation due to their use of specific instrumentation and chemicals.

In present invention, the volumes of the lysis, washing, and dissociation buffers can be easily adjusted, enabling the isolation of RPFs from a single cell using a suitable ultrafiltration membrane or column.

While various aspects of the present invention have been particularly shown and described with reference to the exemplary, non-limiting, embodiments above it will be understood by those skilled in the art that various additional aspects and embodiments may be contemplated without departing from the spirit and scope of the present invention.

### BRIEF DESCRIPTION OF THE DRAWINGS

FIG. 1 depicts an illustration of the workflow and steps involved in isolation of ribosome protected mRNA fragments from ribosome stabilization to sample preparation for small RNA library preparation.

FIG. 2 depicts an illustration of the tangential flow ultrafiltration steps to obtain concentrated monosomes with reduced contaminants.

FIG. 3 depicts an illustration of monosome dissociation and a tangential flow ultrafiltration steps for obtaining ribosome protected mRNA footprints as a filtrate.

FIG. 4 depicts graphical illustrations demonstrating the washing and ultrafiltration processes for removing contaminants, thereby enhancing the purity of monosomes and consequently, ribosome protected mRNA footprints.

FIG. 5 depicts graphical illustrations illustrating monosome dissociation and the ultrafiltration process for obtaining ribosome protected mRNA footprints.

FIG. 6 depicts SYBR Gold stained 15% urea-polyacrylamide gel showing size distribution of isolated RPFs. Ribosome protected footprints are marked by rectangle around 30 nt length. Lane M: DNA ladder (20 ng)-20-100 nt length DNA ladder; Lane 1-4: An aliquot of isolated RPFs at various isolation conditions; Lane-3: One tube reaction where polysomes were washed with lysis buffer twice and then treated with ribonuclease; and further washing to purify monosomes, example-2 for more details; Lane-4: Similar steps to those in lane-1 were followed, except that dissociation was achieved using puromycin, which specifically isolates elongated ribosomes while preventing other RNA-protein complex dissociation; Lane 5-6: Aliquots were taken from the third wash of samples in lanes-1 and 2 to assess the efficiency of the washing steps. Other bands observed in the gel correspond to tRNA and minor impurities. Gel was stained using SYBR Gold Nucleic Acid Stain 1:1000 in TBE buffer for 5 minutes.

FIG. 7A depicts RPFs cleaning statistics by unique mapping to different contaminating RNA types including rRNA, tRNA and snRNA. Bioinformatic analysis was done using RiboToolkit. FIG. 7A demonstrate that the present invention efficiently removes rRNA from isolated RPFs. Fraction of rRNA reads are <0.1% of total reads. No additional rRNA depletion step is required to enrich RPFs.

FIG. 7B depicts statistics of unique read mapping after contaminant removal to demonstrate the efficiency of the present invention.

FIG. 8 depicts the read coverage distribution across the length of isolated ribosome-protected footprints (RPFs). This data demonstrates that the monosome washing and ribosome footprints isolation method outlined in the invention effectively enrich RPFs while maintaining unbiased results.

FIG. 9 depicts the distribution of ribosome-protected footprints (RPFs) across various gene biotypes, such as protein-coding genes, lncRNA, and snRNA. The data shows a high abundance of RPFs linked to protein-coding genes, reinforcing that the RPFs isolation process is unbiased and effectively enrich RPFs without preference.

FIG. 10 depicts the distribution of ribosome-protected footprints (RPFs) across different gene features, such as 5′ UTR, CDS, 3′ UTR, intron, and intergenic regions. The data demonstrate that RPFs are most abundant in the CDS compared to other gene features.

FIG. 11 Depict the distribution of ribosome-protected footprints (RPFs) across different coding frames (0, 1, and 2) within the 5′ UTR, CDS, and 3′ UTR regions. RPFs frames plot by RiboToolkit confirmed the high quality of the Ribo-seq data, demonstrating efficient isolation of RPFs.

FIG. 12 depicts uniform read coverage of ribosome-protected footprints (RPFs) across the ACTB gene, illustrating RPFs mapping in IGV.js. ACTB is used as an example to demonstrate uniform read mapping across the entire transcript, with RPFs clearly confined to the protein-coding sequence.

### DETAILED DESCRIPTION OF THE INVENTION

### Definitions

To facilitate an understanding of the present invention, a number of terms and phrases are defined herein:

The term “ribosome” refers to a cellular organelle responsible for protein synthesis. It consists of ribosomal RNA (rRNA) and proteins and functions by translating messenger RNA (mRNA) into proteins through a process involving transfer RNA (tRNA) and various enzymatic factors.

The term “polysomes” or polyribosomes refers are clusters of ribosomes bound to a single mRNA molecule. These clusters allow multiple ribosomes to simultaneously translate the same mRNA strand, thereby increasing the efficiency of protein synthesis within a cell.

The term “monosomes” refers to single ribosomes that are not associated with polysomes. They typically translate mRNA individually and are involved in the synthesis of proteins. In the present invention, monosomes are generated by digesting exposed or unprotected mRNAs from polysomes. Ribonuclease (such as RNase I) is added to lysate containing polysomes to digest unprotected or exposed mRNA or mRNA outside of translating ribosomes. This enzymatic digestion cleaves the RNA that is not shielded by ribosomes. However, ribosomes or ribosomal RNAs are protected by ribonuclease digestion because of their association with significant number of ribosomal proteins.

The term “cell” refers to the basic structural, functional, and biological unit of all living organisms. Cells are enclosed within a membrane and contain genetic material in the form of DNA. Cells from various organisms are fundamental units of life, exhibiting diverse structures and functions adapted to their specific roles within each organism. Cells can vary widely in size, shape, and function.

The term “ultrafiltration” refers to a separation process used to purify and concentrate molecules based on their size and molecular weight. It involves passing a solution through a semipermeable membrane that selectively allows smaller molecules to pass through while retaining larger molecules. This method is effective for separating proteins, nucleic acids, and other biomolecules from contaminants or unwanted substances, thereby enhancing the purity and concentration of the target molecules.

The term, “permeate” refers to the portion of a sample that passes through the ultrafiltration membrane. This fraction is also referred to as the “filtrate.”

The term “retentate” refers to the portion of a sample that is retained by the ultrafiltration membrane and does not pass through.

The term “tangential flow”, also known as cross-flow filtration, it refers to a filtration method used to separate components in a fluid mixture by passing the fluid along a semi-permeable membrane.

The term “semi-permeable membrane” refers as a selective barrier that allows certain molecules or ions to pass through while restricting others.

The term “ribonuclease (RNase)” refers to an enzyme that catalyzes the degradation of RNA into smaller components. This enzyme cleaves the phosphodiester bonds within RNA molecules.

The term “RNase-I” refers to an endoribonuclease that preferentially hydrolyzes single-stranded RNA to nucleoside 3′-monophosphates. In the present invention we refer RNase-I as a ribonuclease.

The term “DNase I” refers as an enzyme that specifically degrades DNA by cleaving the phosphodiester bonds between nucleotides. This endonuclease acts on double-stranded and single-stranded DNA, converting it into smaller fragments.

The term “RPFs” refers to ribosome protected mRNA footprints or fragments. This term is invariably used in the present invention.

### GENERAL EMBODIMENTS OF THE INVENTION

The present invention relates to a new method for isolating RPFs directly from ribonuclease treated total lysate. In the present invention previously used time-consuming and expensive methods such as monosome isolation by sucrose cushion or polysome fractionation has been avoided. Further, it doesn't use phenol-chloroform extraction step to avoid highly toxic chemicals. Present invention reduces processing time, instrumentation and amount of starting reagent. These aspects make the present invention inexpensive and very simple for isolation of RPFs.

In one embodiment, cell lysis solution comprising about 0.05%-2.0% (w/v) of detergent from the group combination thereof. Tween 20, Triton X-100, Sodium deoxycholate, Nonident P-40, IGEPAL CA-630, SDS, and buffering agents combination thereof Tris, HEPES consisting of 0.05-0.20 M solution and alkaline earth metal halides and combination thereof CaCl2), MgCl2 consisting of 0.05-10 M solution, salt solutions and combination thereof NaCl, KCl consisting of 0.0050-1000 M solution and the combination thereof at a pH of about 6.0-10.0 and glycerol.

In one embodiment, the cell lysis solution comprising glycerol, a surfactant the glycol family such as polyalkylene glycol or Triethylene glycol or polyethylene glycol. The lysis buffer can consist of the closest analog to it used commonly propylene glycol (propane 1,2-diol), propane-1,2-diol, propane-1,3-diol, 1,1,1-tris-(hydroxymethyl) ethane (THME), and 2-ethyl-2-(hydroxymethyl)-propane-1,3-diol (EHMP). The lysis buffer used in the present invention advantageously contain glycerol in an amount between 0.05% and 60%, in particular between 0.5 and 40%, in particular between 2 and 20% and in particular between 5 and 60%. Alternatively, sucrose and trehalose can be used for efficient cell lysis including ribosome stabilization, and preservation. It is believed that the presence of glycerol in the lysis buffer is advantageous as it improves cell lysis and preserve translating ribosome structure which increases the yield of ribosome protected footprints (RPFs).

The starting material for the present invention is eukaryotic cells. In the present invention lysis buffer composition is tailored to the specific eukaryotic cells however, it will change with the requirements of different cell types or species. Lysis buffer composition variability is crucial for achieving efficient cell lysis and subsequent extraction of cellular contents. For example, mammalian cells generally need a different buffer composition than bacterial cells because their membranes contain cholesterol and glycoproteins. Therefore, for plant, fungal, and bacterial cells, the composition of the lysis buffer must be adjusted to include agents that can effectively break down the cell wall without disturbing the polysome composition and their locations on the mRNA. After cell lysis sample was centrifugated at 10,000 g at 4° C. for 10 minutes to remove cell debris, unbroken cells, and nuclei. This step is crucial for monosome or polysome purification, as solid particles in the cell lysate can clog the ultrafiltration device during monosome or polysome washing. Alternatively, if a homogeneous and clear lysate is obtained, it can be directly applied to the column for RPFs isolation.

In one embodiment, the present invention is demonstrated using MCF7 cells. Since the function of ribosomes in protein synthesis is similar across all domains of life, with only minor structural and mechanistic differences, this invention is applicable to other cell lines, species, and biological samples.

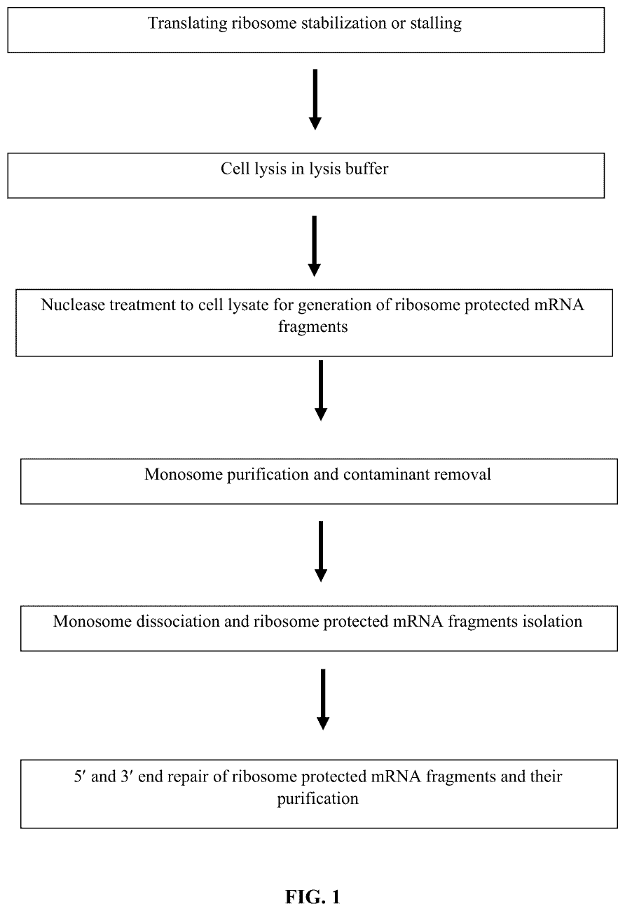

In one embodiment, stabilization of ribosomes is necessary to obtain specific mRNA translation information, such as initiation, elongation, termination, or steps preceding initiation or following termination. Furthermore, inhibitors may be applied before, during, and after cell lysis, as well as during the ribonuclease digestion step, to selectively identify specific stages of translation. Ribosome stabilization and cell lysate preparation steps are depicted in FIG. 1.

In one embodiment, monosomes are generated from polysomes by ribonuclease digestion step. Ribonuclease digestion step is also known as “nuclease protection assay” where sandwiched mRNA fragment is protected by ribosome from ribonuclease digestion. Monosomes consist of single ribosome with or without mRNA. Ribosomes with mRNA are called stalled, initiating and elongating. Ribosomes without mRNA are called vacant ribosomes. Ribonuclease (RNase-I) is an endoribonuclease that selectively hydrolyzes single-stranded RNA. Ribosome protects 20-35 nt length mRNA from ribonuclease digestion and length of RPFs depends on species of ribosome, ribosome stabilization conditions, ribosome conformation, composition of cell lysis buffer and ribonuclease digestion conditions. In the present invention, a specific cell lysis buffer composition was employed to stabilize ribosomes and generate RPFs of uniform length. Composition buffer comprising of magnesium, sodium chloride, potassium chloride, triton-X 100 and sodium deoxycholate or their combinations.

In one embodiment, the present invention was demonstrated using RNase-I ribonuclease however, other ribo-endonucleases can also be used.

According to one embodiment, during the nuclease protection assay, DNase enzyme (DNase-I) was added to degrade genomic DNA, thereby eliminating false positive signals that could result in inaccurate gene expression measurements or the erroneous identification of new coding regions. Further, it reduces the viscosity of the total lysate, thereby preventing clogging of the semipermeable membrane during ultrafiltration. DNase treatment comprising of similar conditions and buffer composition to that of ribonuclease protection assay.

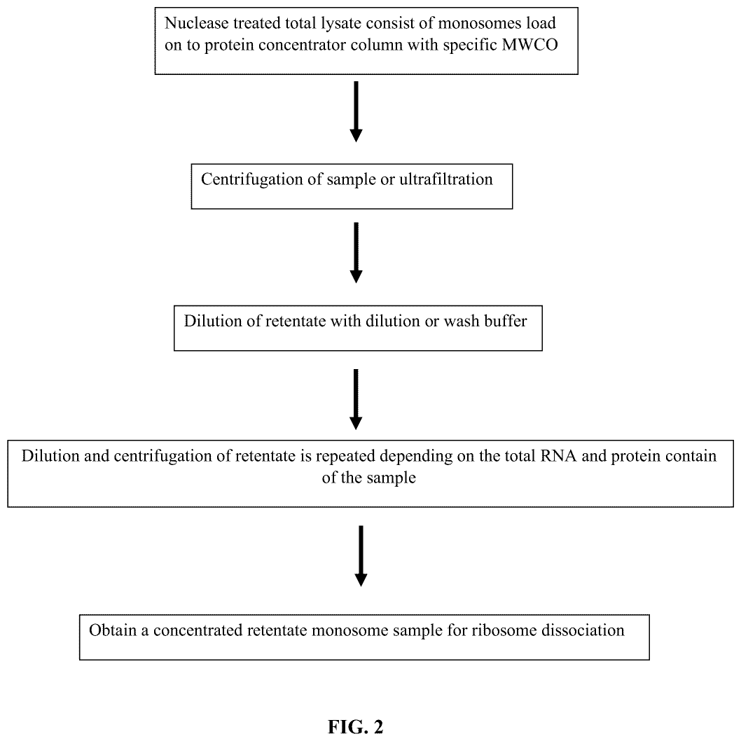

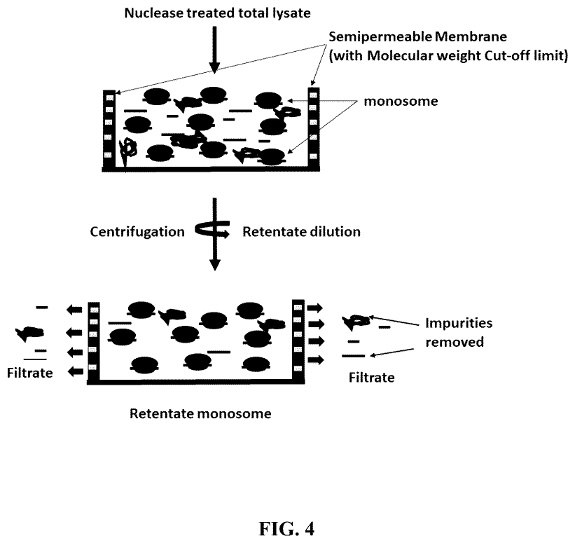

In one embodiment for monosome purification by tangential flow ultrafiltration, the nuclease treated sample (total lysate) was centrifuged in a column against a semipermeable membrane allowing molecular weight cut-off (MWCO) limit. The components of the solution with molecular weights smaller than MWCO limit would pass through the semipermeable membrane during centrifugation, while higher than MWCO limit cellular components are retained. During centrifugal ultrafiltration process the monosomes are retained due to their higher MWCO limit and concentrated because dilution buffer and contaminant passes through the semipermeable membrane. Semipermeable membrane column which has MWCO limit between higher than 50 kDa and about 4 mDa may be used for monosome purification and concentration. Prior to centrifugal ultrafiltration, nuclease treated sample (total lysate) is diluted by 1-30-fold to obtain low solute concentration with dilution buffer. Subsequent ultrafiltration concentrates the diluted sample to a predetermined volume which lead to increase in the concentration of monosome fraction and reduced concentration of contaminants. Sample dilution and ultrafiltration sequential steps are repeated 1-10 times depending on the starting RNA content in the total lysate and or until the contaminant are reduced to lowest level. This process, known as “Discontinuous diafiltration,” involves repeated steps of concentrating the sample through ultrafiltration and rediluting it with buffer until the desired yield and purification are achieved. Diafiltration may be either continuous or discontinuous depending on the setup of the experiment. In the last step monosomes are concentrated to its lowest volume which depend on the columns dead volume limit of ultracentrifuge membrane. In the end retained monosome are now at its original level and permeable components particularly contaminants have been washed out. During these dilution and ultrafiltration steps monosomes are not dissociated which protect the mRNA footprints. The use of ultrafiltration column aspect makes the present invention simpler and potentially more economical than prior art. FIG. 2 and FIG. 4 depicts the dilution and ultrafiltration steps to reduce the contaminants concentration.

The ultrafiltration membrane should be composed of a low-binding material to minimize adsorptive losses and must be durable and chemically compatible with the buffers used. Several suitable membranes are commercially available, including cellulose acetate, polysulfone, polyethersulfone, and polyvinylidene difluoride. Preferably, the choice of membrane material depends on the specific RPFs isolation conditions and reagents used in the process.

For polysome and monosome purification, the optimal membrane pore size or molecular weight cutoff (MWCO) may be determined according to manufacturer instructions. The size of the ultrafiltration column is determined by the characteristics of the starting material, volume, and isolation conditions. Present invention uses 100 kDa MWCO, 0.5 ml column from Millipore Sigma for RPFs isolation.

In one embodiment, the higher the amount of input RNA, the larger the column volume required to accommodate it. Additionally, the volume of the monosome-concentrated sample is further reduced by transferring it to a small size ultrafiltration column with use of specific MWCO limit and approximately 25 μL dead volume. This step help reduces the volume of retentate to lowest workable volume where sample is concentrated to smaller quantity such as between about 25 ul and about 50 ul.

In one embodiment, nuclease treated total lysate may be diluted between 1-fold and 30-fold with lysis buffer. Dilution buffer component is magnesium with concentration is between about 2.5 and about 5 mM to reduce non-specific RNA-RNA, RNA-protein and RNA-DNA interactions without dissociating monosomes. Additionally, potassium or sodium chloride is known to affect RNA-protein interaction. Hence, diluting nuclease treated sample with dilution buffer reduces non-specific RNA interactions and dilute the contaminant to low concentration. Diluted sample is centrifugated by maintaining centrifugal temperature between about 4° C. and about 20° C. to obtain concentrated monosome sample by removing the contaminant. By keeping centrifugal temperature low helps to avoid RNA degradation by RNases. Contaminant RNA are those that are not associated with or protected by ribosomes which reduce the depth of useful reads in next generation sequencing. One major problem of RNA sequencing is the presence of ribosomal RNA (rRNA) contaminant in the sequencing sample. rRNA is the most abundant molecule present in total RNA, which represents around 90-95% of isolated RNA sample. Further, several other RNAs such as transfer RNA (tRNA), microRNA (miRNA), SnoRNA etc. present in variable quantity (0.1-5%) in the RNA sample. Abundant rRNA and several unwanted RNA are carried over into the library construction and sequencing, thereby low signal-to-noise ratio making it difficult to detect useful reads. During ribonuclease digestion, rRNA and other RNA are digested into numerous RNA fragments of variable length. Several rRNA depletion technologies are available for rRNA depletion. However, in rRNA depletion, rRNA oligo may cross-hybridize with non-target RNAs, thereby resulting in a non-specific removal of RNA under study causing artifacts in final data. Therefore, by removing contaminant rRNAs and other unwanted RNA by diluting and concentration by centrifugal filtration maximize the number of useful reads available for investigation. Furthermore, monosomes are retained in the solution as a retentate. “Retentate” refers to a sample component that do not pass through the ultrafiltration membrane. In the present invention, “contaminant” refers to RNAs and their fragments that are not translated by ribosomes, as well as DNA and their fragments. Steps for monosome purification are depicted in FIG. 2 and FIG. 4.

In one embodiment, total lysate containing polysomes may be diluted between 1-fold and 30-fold with a lysis buffer. The diluted sample was centrifugated 1-10 times to remove translational components such as ternary complex, tRNA, GTP, ATP, amino acids, elongating factors etc. to stop the ongoing translation during nuclease treatment. Ribosomes or polysomes possess inherent translation capability; therefore, if all necessary components are present, they will continue translating along the mRNA. Ribosome protects ˜30 nt length of mRNA however, continued translation during nuclease treatment may generate <30 nt length RPFs. 5-End of digested mRNA is exposed during continue movement of ribosomes over digested 3-end because of translation. Therefore, the exposed 5′-end of mRNA is redigested, resulting in RNA fragments smaller than 30 nucleotides in length. Hence, removing translational components from the total cell lysate helps improve the uniformity of RPFs and reduce <30 nt length RPFs fraction by stalling ribosomes at one location on mRNA. Additionally, this step removes free-floating tRNA from the sample, thereby reducing fragmented tRNA reads in the final output. After washing the polysomes, total lysate was treated with ribonuclease and then again washed 1-20 times to remove contaminants generated during treatment. Hence, washing the sample before and after ribonuclease treatment can improve the uniformity and quality of RPFs. Therefore, reducing fragmented rRNA and tRNA reads enhances the proportion of useful sequencing reads, thereby increasing the depth of sequencing for identifying rare RNA molecules.

In one embodiment, during monosome purification dilution and ultrafiltration were repeated to remove impurifies or contaminants from nucleases digested and undigested total lysate. However, care was taken that the dilution factor is not extreme and the ionic concentrations of dilution buffer is suitable to keep the ribosomes or monosomes intact. The buffers used in the monosome purification are designed to mimic the ionic conditions within the cell, providing physiological conditions that supports the monosome stability and avoid nonspecific interaction. For ribosome subunits, the association constant is usually in the range of 1 nM to 10 nM range reflecting the strong binding, however, it can vary depending on the experimental conditions and starting total RNA amount. Hence, for ribosome footprints isolation starting with 1.5 ug of total RNA, the monosomes can tolerate a dilution range of 1-1000×. Hence, maintaining proper magnesium ion concentrations, appropriate ionic strength and temperature control all contribute towards the preserving the integrity of monosomes during washing steps.

Through persistent experimentation, we iteratively refined our method by adjusting conditions such as lowering the concentration of starting RNA in the total lysate because at higher concentration of total lysate the column was clogging. Further, at higher quantity of total lysate RNA degradation was not generating homogenous or good quality of PRFs in the column. During monosome purification ionic concentration of KCl, NaCl and Mg was titrated to get the right combination of washing buffer to avoid monosome dissociation. Furthermore, washing steps were adjusted to enough to remove impurities without dissociating monosomes. Each of the above stapes were variably affecting the quantity and quality of the ribosome footprints. This final process in the application is yielding superior results in short time with less instrumentation compared to previous methods.

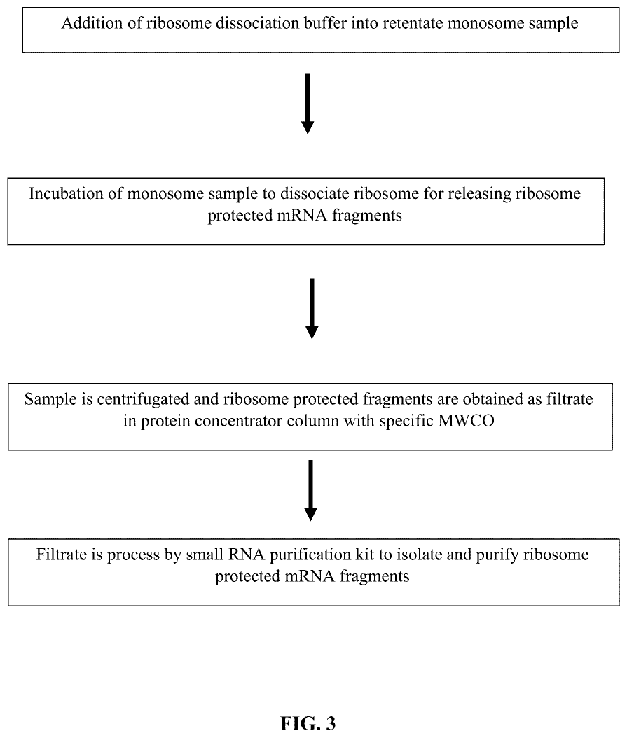

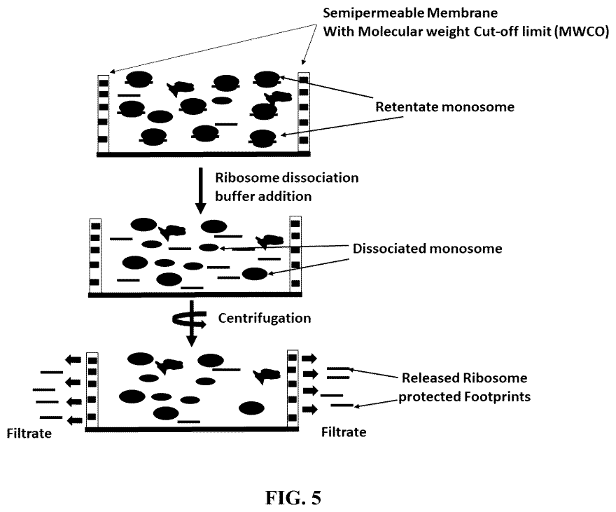

In one embodiment, ribosome protected mRNA fragments are released into solution from monosome by dissociating them. Concentrated monosome sample is treated with about 1 mM or higher concentration of EDTA in dilution buffer at 4° C. for 15-30 min. EDTA binds to metal ions with a +2 charge (e.g. magnesium), thereby making them unavailable for interactions which stabilize RNA structure. Therefore, EDTA treatment destabilized RNA-RNA interactions which leads to monosome dissociation into large and small subunits concomitantly release protected mRNA fragment (RPFs). Puromycin, in combination with a high concentration of potassium chloride, is known to cause the dissociation of elongating ribosomes. In the present method, the retentate or concentrated monosomes are treated with >2 mM puromycin in >20 mM potassium chloride and approximately 2.5 mM magnesium, at 37° C., or at room temperature, or using a combination of these conditions to dissociate monosomes. This treatment specifically releases elongating mRNA by targeting the elongating ribosomes or monosomes. However, EDTA may dissociate other RNA-RNA and RNA-protein complexes which may contaminate RPFs with RNA footprints protected by other proteins. Released RPFs are collected as permeate that passes through the ultrafiltration membrane and is also termed the filtrate. Ultrafiltration semipermeable membrane whose MWCO limit is higher than 30 kDa or in combination with other MWCO limit may be used to selectively isolate specific length of RPFs. Thus, the method provided by present invention provides significant reduction in several complex steps for isolation of RPFs. The purity of isolated RPFs may be ascertained by spectrophotometrically by measuring the A260/A280 and A260/A230 absorption ratio. If needed, additional purification of RPFs can be performed using 15% polyacrylamide TBE-urea gel electrophoresis. Steps for monosome dissociation and RPFs isolation are depicted in FIG. 3 and FIG. 5.

RPFs purified by tangential flow ultrafiltration can be used directly or subjected to additional purification, depending on the contamination level, the intended application, and the specific requirements of the sequencing and library preparation methods.

In one embodiment, isolated RPFs are end repaired to make compatible with several small RNA library preparation kits. Ribonuclease digestion (such as RNase-I) produce RPFs with a 52-hydroxyl and a 32-phosphate ends via nucleoside 22, 32-cyclic monophosphate intermediates. RPFs were treated with T4 PNK in the presence or absence of ATP to generate 52-phosphate and 32-hydroxyl ends. T4 PNK treatment reverse the phosphate position and removes any 2232 cyclic monophosphate making RPFs compatible to many small RNA sequencing library preparation kits. In the same reaction RNase free DNase-I is added to hydrolyze contaminating genomic DNA fragments isolated with RPFs. DNase treatment removes any remaining genomic DNA fragments that are copurified with RPFs.

In one embodiment, present invention relates to a kit for isolation and purification of ribosome protected mRNA fragments or footprints from a biological sample. The kit comprising, (a) lysis buffer for efficient cell lysis and ribosome stabilization; (b) dilution or wash buffer for reducing non-specific RNA-RNA interactions and inhibiting RNase activity; (c) dissociation buffer to dissociate monosomes and release RPFs; (d) 5′ and 3′ end repair buffer for 5′ phosphorylation and 3′ dephosphorylation of RPFs, which also maintains the activity of DNase I enzyme for effective DNA degradation; (e) molecular weight cut-off (MWCO) columns or plate for monosome purification and RPFs isolation; and (f) instructions for isolation of RPFs.

Exemplary advantages of the present invention is to provide a method and composition that isolate RPFs with fewer contaminants in a simple, single-column purification step. The present invention allows purification of RPFs without any specialized instrumentation compared to available methods. This is a significant improvement compared to prior art methods.

All technical and scientific names and terms used herein have the same meaning as commonly understood in the research field.

The following example demonstrates certain aspects of the described methods and its advantageous results. The materials, methods and examples are illustrative purpose only and not intended to be limiting the scope.

### Example 1

### Materials

Cell Lysis or ribosome stabilization buffer: 10 mM Tris HCl pH 7.4; 50 mM KCl; 10 mM MgCl2; 0.1% Triton-X 100; 4 units DNase, 40 units RNase inhibitor murine and 1% sodium deoxycholate (use fresh solution).

Dilution or wash buffer: 20 mM Tris HCl pH 7.4; 200 mM KCl and 3.5 mM MgCl2.

EDTA-Dissociation buffer: 100 mM Tris HCl pH 7.4, 500 mM KCl, 2.5 mM MgCl2 and 30 mM EDTA pH:8.0.

Puromycin-Dissociation buffer: 100 mM Tris HCl pH 8.0, 500 mM NaCl, 2 mM MgCl2 and 8 mM puromycin.

End repair buffer: 70 mM Tris HCl pH 7.4; 10 mM MgCl2; and 5 mM DTT.

RNA extraction buffer: 300 mM KOAc, 1 mM EDTA and 0.25% w/v SDS.

### Brief Description of Steps Involved in the Purification of RPFs

MCF7 cells were cultured in Dulbecco's Modified Eagle Medium (DMEM) supplemented with 10% Fetal Bovine Serum (FBS) and 1% penicillin-streptomycin in a 5% CO2 incubator at 37° C. The cells were grown to 60-70% confluence in 6-well plates, which is approximately 1×10⁶ cells per well at the time of lysis. The cell media was aspirated, and the cells were washed with 2 ml of cold 1×PBS per well. To lyse the cells, 200 μl of cold lysis buffer (containing 5% glycerol, 1.5 units of DNase, and 10 units of RNase inhibitor) was added to each well. The cells were scraped and collected into a non-stick RNase-free 1.5 ml microcentrifuge tube, which was then placed on ice for 10 minutes to ensure complete lysis. The sample was centrifuged at 10,000×g at 4° C. for 10 minutes, and the supernatant was transferred to another pre-chilled, non-stick RNase-free 1.5 ml microcentrifuge tube. The supernatant was diluted 1:10 in water, and its absorbance (AU) was measured at A260 using the nucleic acid function of a Nanodrop spectrophotometer. A 1:10 dilution of lysis buffer in water was used as the blank for subtraction. Aliquots of the sample or supernatant were either flash-frozen in liquid nitrogen and stored at −80° C. or proceeded with nuclease digestion. A total lysate with an absorbance of 0.25 AU, corresponding to approximately 1.5 μg of RNA, was used for RNase I digestion. RNase I (1.5 units) and DNase (1.5 units) were added to each 250 μl digestion reaction containing 1.5 μg of total RNA. The final volume of the RNase I digestion reaction was adjusted to 250 μl by adding lysis buffer. The reaction was incubated at 25° C. for 45 minutes in a thermomixer set to 300 rpm. After incubation, the nuclease-treated lysate was transferred to a 0.5 mL ultra-centrifugal filters with 100 kDa molecular weight cut-off limit and spun at 14,000×g for 20 min at 4° C. About 25 μl of retentate was diluted by adding 500 μl washing or dilution buffer and mixed gently by pipetting and spun at 14,000×g for 20 min at 4° C. Retentate dilution and centrifugation were repeated three times to enhance the purity of the RPFs. In the final step, the retentate sample was concentrated to approximately 25 μL by centrifugation.

In the retentate sample 120 ul of EDTA containing dissociation buffer was added and mixed by pipetting several times and kept at 4° C. for 20 min with interval mixing. Sample was spun at 14,000×g for 20 min at 4° C. and collected 120 μL filtrate in a new RNase free tube. RNA clean & concentrator-5 spin column from Zymo was used for isolation of RPFs. RNA ethanol binding buffer was prepared by mixing equal volumes of RNA binding buffer from Zymo kit with 100% Ethanol. In a typical experiment, 120 μL of filtrate was mixed with 240 μL of RNA ethanol binding buffer by pipetting. To this mixture, 360 μL of ethanol was added and the resulting 720 μL sample mixture was applied to an RNA Clean & Concentrator-5 spin column. Following this, 400 μL of RNA Prime Buffer was passed through the column. The RNA Clean & Concentrator-5 column was washed according to the instructions provided in the Zymo RNA Clean & Concentrator-5 kit. RPFs were then eluted in 15 μL of RNase-free water and quantified using a Nanodrop spectrophotometer or Qubit microRNA assay kit.

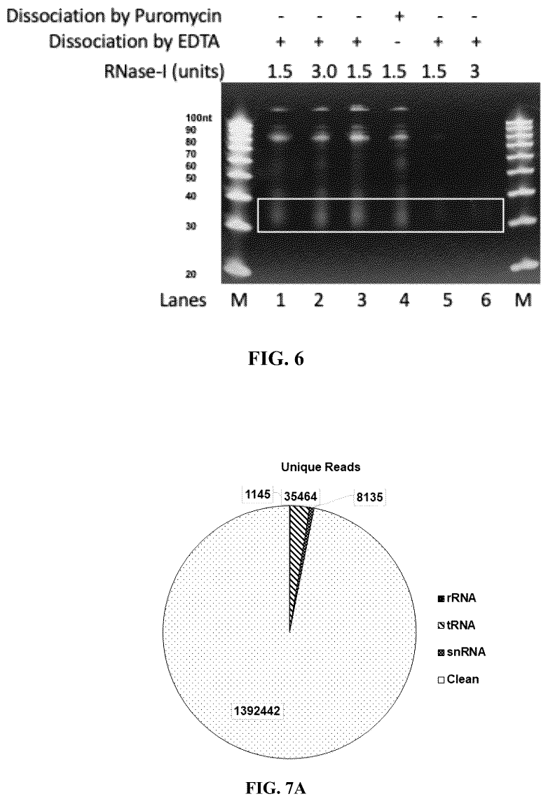

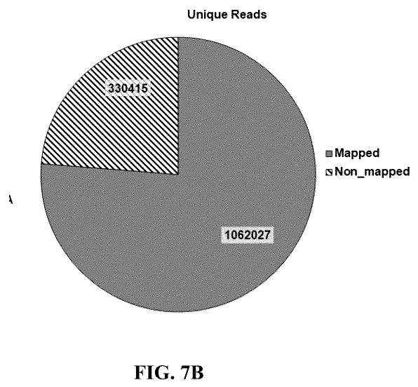

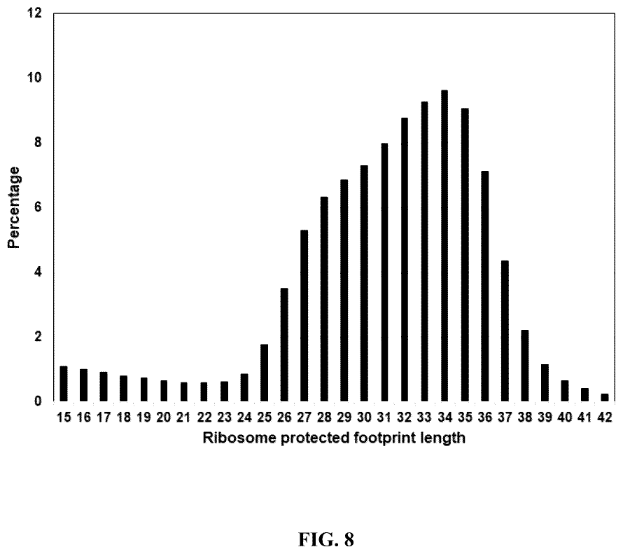

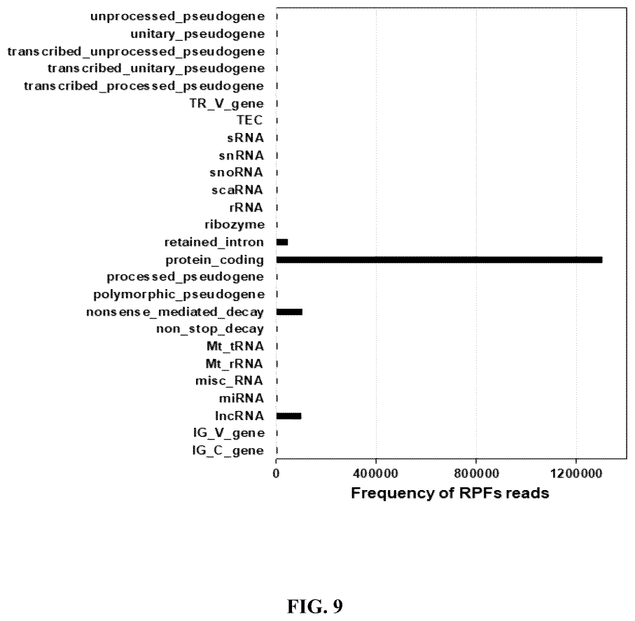

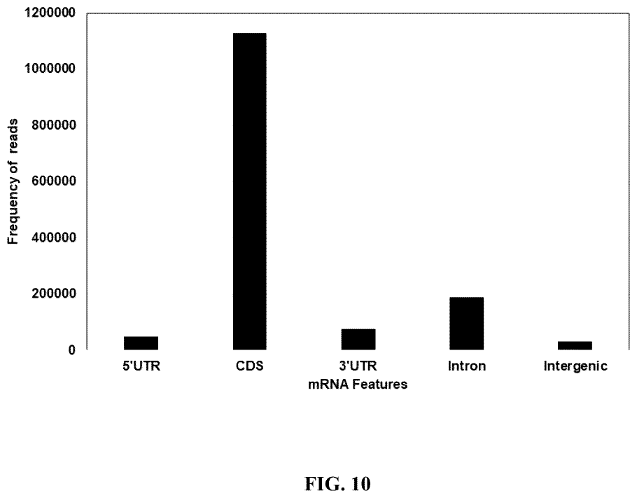

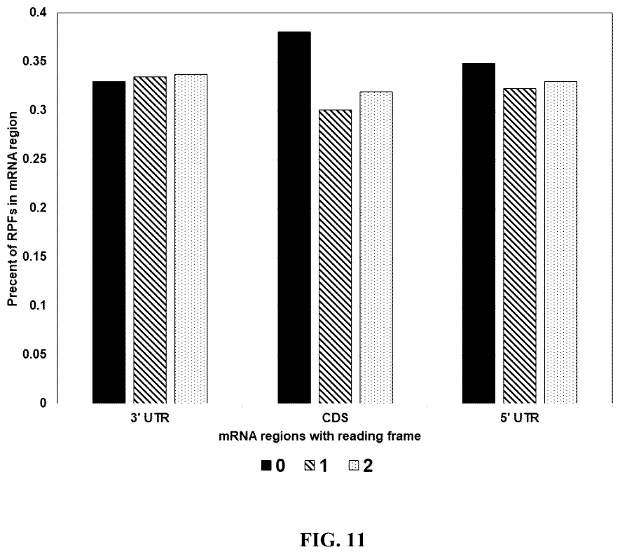

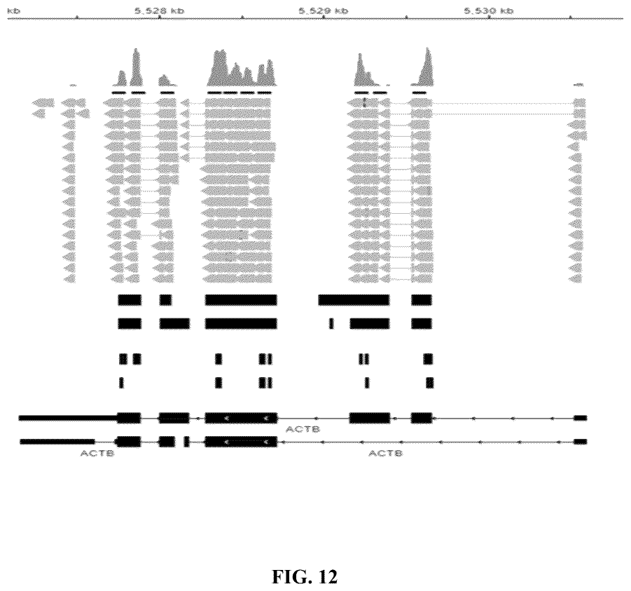

Isolated RNA was denatured in a 1:1 mixture of 2×RNA loading dye at 70° C. for 5 minutes. RPFs were then separated and size-selected on a 15% polyacrylamide TBE-urea gel electrophoresis at 200V for 45 minutes. The gel was stained with SYBR Gold stain and visualized. The 25-35 nucleotide band was excised from the gel. Size-selected RPFs were extracted by freezing the gel in RNA extraction buffer at −80° C. for at least 1 hour, followed by overnight rotation at 4° C. with 40 units of SUPERase*In RNase inhibitor. RNA was precipitated with 500 μL isopropanol and 1.0 μL GlycoBlue at −80° C. for 2-3 hours. The RNA was pelleted by centrifugation at 14,000×g for 30 minutes at 4° C., then washed once with 80% ethanol, air-dried, and resuspended in 15 μL of RNase-free water. RNA concentration was measured using a Qubit Flex fluorometer with Qubit microRNA assay Kit. Gel electrophoresis results shown in the FIG. 6, Lane 1-2 demonstrate that present invention method work efficiently in isolating RPFs. FIG. 6, Lane 6-7 demonstrates that three washing steps are sufficient to remove contaminants and generate high-quality reads.

### Example 2

MCF7 cell culture and total lysate preparation were carried out according to the protocol described in Example 1. For the experiment, a total lysate with an absorbance of 0.25 AU at A260, corresponding to approximately 1.5 μg of RNA was used. The lysate volume is adjusted to 250 μL by adding lysis buffer and then transferred to a 0.5 mL ultra-centrifugal filters with 100 kDa molecular weight cut off limit and spun at 14,000×g for 20 min at 4° C. ˜25 ul Retentate is diluted by adding 500 μl lysis buffer and mixed gently by pipetting and spun at 14,000×g for 20 min at 4° C. In approximately 25 μL of retentate, RNase I (1.5 units), DNase (1.5 units), and RNase inhibitor (10.0 units) are added to prepare a 250 μL digestion reaction using lysis buffer. The reaction was incubated at 25° C. for 45 minutes in a thermomixer set to 300 rpm. After 45 minutes, the reaction is centrifuged at 14,000×g for 20 minutes at 4° C. The ˜25 μL retentate is then diluted by adding 500 μL of washing or dilution buffer, gently mixed by pipetting, and centrifuged again at 14,000×g for 20 minutes at 4° C. This process of retentate dilution and centrifugation is repeated three times to enhance the purity of the RPFs.

In the retentate sample 120 μl of EDTA containing dissociation buffer was added and mixed by pipetting several times and kept at 4° C. for 20 min with interval mixing. Sample was spun at 14,000×g for 20 min at 4° C. and collected 120 μl filtrate in a new RNase free tube. The RNA Clean & Concentrator-5 spin column from Zymo was used for RNA isolation as described in Example 1 and the RPFs were eluted in 15 μL of water. Isolated RNA was denatured in a 1:1 mixture of 2×RNA loading dye at 70° C. for 5 minutes. RPFs were then separated on a 15% polyacrylamide TBE-urea gel electrophoresis at 200V for 45 minutes. The gel was stained with SYBR Gold stain and visualized. Gel electrophoresis results are shown in the FIG. 6, Lane 3 demonstrates that the present invention method works efficiently in isolating RPFs when polysomes are washed prior to ribonuclease treatment.

### Example 3

All the steps are followed as given in the Example 1 except the dissociation of monosome was done using puromycin. In a typical experiment 120 μl of puromycin containing dissociation buffer having RNase inhibitor (10 units) were added, mixed by pipetting several times and kept at 25° C. for 20 min with 1000 rpm. Sample was spun at 14,000×g for 30 min at 4° C. and collected 120 μL filtrate in a new RNase free tube. RPFs were isolated by Zymo kit given in the Example 1 and eluted in 15 μL water. Isolated RNA is denatured in a 1:1 mixture of 2×RNA loading dye at 70° C. for 5 minutes. RPFs were then separated on a 15% polyacrylamide TBE-urea gel electrophoresis at 200V for 45 minutes. The gel was stained with SYBR Gold stain and visualized. Gel electrophoresis results shown in the FIG. 6, Lane-4 demonstrates the highly efficient and rapid isolation of RPFs using puromycin, which specifically dissociates elongating ribosomes.

### Example 4

Brief description of steps involved in the 52 and 32 end repair of RPFs RPFs were end-repaired in 50 μL of end repair buffer containing 2 μL RNase Inhibitor Murine (80 units), 3 μL T4 Polynucleotide Kinase (30 units), RNase-free water, and 15 μL of RPFs isolated from urea gel or ultrafiltration over a period of about 60 minutes at 37° C. To the 50 μL reaction, 100 μL of RNA binding buffer was added and mixed by pipetting several times. Next, 400 μL of 100% ethanol was added to the same sample and mixed again. The RPFs were then recovered using an RNA Clean & Concentrator-5 spin column (from the Zymo RNA Clean & Concentrator-5 kit) as described in Example 1. The RPFs were eluted in 10 μL of nuclease-free water, and the concentration of end-repaired RPFs was determined using a Qubit miRNA assay kit.

### Brief Description of Small RNA-Seg Library Preparation of RPFs

The QIASeq miRNA Library Kit was used to generate small RNA libraries from RPFs. For library preparation, 10 ng of gel-purified RPFs was utilized. The library was then amplified using 6 PCR cycles. Purification of the library was performed using TBE-PAGE as specified in the QIASeq miRNA Library Kit. During gel purification, contaminant bands generated in the library due to tRNA and adapters were removed. Samples were sequenced using the Illumina HiSeq 2000 with NextSeq 1000/2000 P2 Reagents (100 Cycles) v3 flow cell. QIAseq miRNA Library Kit-derived libraries were sequenced using 75 bp single reads, with approximately 5 million reads allocated per sample.

### Ribo-Seq Analysis

The QiAseq miRNA adapter removal steps were performed according to the QiAseq miRNA analysis protocol. Read sequences were initially aligned to rRNAs, tRNAs, and snRNAs to exclude reads originating from these RNA types. This alignment was performed using Bowtie v1.2.2 with a maximum of two mismatches allowed (−v 2) by default. Cleaned RPF sequences were then mapped to the reference hg19 human genome using STAR v2.7.3a with the following default parameters: -outFilterMismatchNmax 2, -quantMode TranscriptomeSAM GeneCounts, -outSAMattributes MD NH, and -outFilterMultimapNmax 1. The uniquely mapped RPFs were subsequently aligned against protein-coding transcripts using Bowtie v1.2.2 with default parameters -a -v 2. Coding frame distribution analyses for Ribo-seq quality evaluation was conducted using riboWaltz v1.1.0. Sequenced libraries were analyzed using published RiboToolkit platform to identify: 1) unique reads coverage, 2) read length distribution of RPFs, 2) GeneBiotype, 3) Reads coverage in CDS and UTR regions, 4) RPFs frame distribution, and 5) read coverage of RPFs over all identified genes.

Other aspects, objects and advantages of the present invention can be obtained from a study of the drawings, the disclosure and the claims.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PATENTS

(2014). US 2014/0288097 A1
(2017) U.S. Pat. No. 9,677,068 B2
(2017) WO 2017/013547 A1
(2013) US 2013/0096012 A1 PUBLICATIONS
1. Mouilleron H, Delcourt V, Roucou X. Death of a dogma: eukaryotic mRNAs can code for more than one protein. Nucleic Acids Res. 2016; 44(1):14-23.
2. Kochetov A V. Alternative translation start sites and hidden coding potential of eukaryotic mRNAs. Bioessays. 2008; 30(7):683-691.
3. Menschaert G, Van Criekinge W, Notelaers T, et al. Deep proteome coverage based on ribosome profiling aids mass spectrometry-based protein and peptide discovery and provides evidence of alternative translation products and near-cognate translation initiation events. Mol Cell Proteomics 2013; 12(7):1780-1790.
4. Scheper G C, van der Knaap M S, Proud C G. Translation matters: protein synthesis defects in inherited disease. Nat Rev Genet. 2007; 8(9):711-723.
5. Gonskikh Y, Polacek N. Alterations of the translation apparatus during aging and stress response. Mech Ageing Dev. 2017; 168:30-36.
6. Liu Y, Beyer A, Aebersold R. On the Dependency of Cellular Protein Levels on mRNA Abundance. Cell. 2016; 165(3):535-550.
7. Fesenko I, Kirov I, Kniazev A, Khazigaleeva R, Lazarev V, Kharlampieva D, Grafskaia E, Zgoda V, Butenko I, Arapidi G, Mamaeva A, Ivanov V, Govorun V. Distinct types of short open reading frames are translated in plant cells. Genome Res. 2019; 29(9):1464-1477.
8. Kochetov A V, Ponomarenko M P, Frolov A S, Kisselev L L, Kolchanov N A. Prediction of eukaryotic mRNA translational properties. Bioinformatics. 1999; 15(7-8):704-712.
9. Kochetov A V, Allmer J, Klimenko A I, Zuraev B S, Matushkin Y G, Lashin S A. AltORFev facilitates the prediction of alternative open reading frames in eukaryotic mRNAs.

### Bioinformatics. 2017; 33(6):923-925.

10. Coon J J, Syka J E, Shabanowitz J, Hunt D F. Tandem mass spectrometry for peptide and protein sequence analysis. Biotechniques. 2005; 38(4):519, 521, 523.
11. Ingolia N T, Ghaemmaghami S, Newman J R, Weissman J S. Genome-wide analysis in vivo of translation with nucleotide resolution using ribosome profiling. Science. 2009; 324(5924):218-223.
12. Steitz J A. Polypeptide chain initiation: nucleotide sequences of the three ribosomal binding sites in bacteriophage R17 RNA. Nature. 1969; 224(5223):957-964.
13. Wolin S L, Walter P. Ribosome pausing and stacking during translation of a eukaryotic mRNA. EMBO J. 1988; 7(11):3559-69.
14. Calviello L, Mukherjee N, Wyler E, et al. Detecting actively translated open reading frames in ribosome profiling data. Nat Methods. 2016; 13(2):165-170.
15. Bazzini A A, Johnstone T G, Christiano R, et al. Identification of small ORFs in vertebrates using ribosome footprinting and evolutionary conservation. EMBO J. 2014; 33(9):981-993.
16. Ingolia NT. Ribosome Footprint Profiling of Translation throughout the Genome. Cell. 2016; 165(1):22-33.
17. Hornstein N, Torres D, Das Sharma S, Tang G, Canoll P, Sims P A. Ligation-free ribosome profiling of cell type-specific translation in the brain. Genome Biol. 2016; 17(1):149.
18. Guo H, Ingolia N T, Weissman J S, Bartel D P. Mammalian microRNAs predominantly act to decrease target mRNA levels. Nature. 2010; 466(7308):835-840.
19. Clamer M, Tebaldi T, Lauria F, et al. Active Ribosome Profiling with RiboLace. Cell Rep. 2018; 25(4):1097-1108.e5.
20. Yusupova G, Yusupov M. Ribosome biochemistry in crystal structure determination.

RNA. 2015 April; 21(4):771-3.
21. Heiman M, Kulicke R, Fenster R J, Greengard P, Heintz N. Cell type-specific mRNA purification by translating ribosome affinity purification (TRAP). Nat. Protoc. 2014; 9(6):1282-1291.
22. Gerashchenko M V, Gladyshev V N. Translation inhibitors cause abnormalities in ribosome profiling experiments. Nucleic Acids Res. 2014; 42(17):e134.
23. Liu Q, Shvarts T, Sliz P, Gregory R I. RiboToolkit: an integrated platform for analysis and annotation of ribosome profiling data to decode mRNA translation at codon resolution. Nucleic Acids Res. 2020 Jul. 2; 48(W1):W218-W229.

Although the present invention and its advantages have been described in detail, it should be understood that various changes, substitutions, and alterations can be made herein without departing from the spirit and scope of the invention as defined by the appended claims. Moreover, the scope of the present application is not intended to be limited to the embodiments of the process, means, methods and steps described in the specification. As one of ordinary skill in the art will readily appreciate from the disclosure of the present invention, processes, compositions, means, methods, or steps, presently existing or later to be developed that perform substantially the same function or achieve substantially the same result as the corresponding embodiments described herein may be utilized according to the present invention.

1. A method for isolation and purification of ribosome protected mRNA fragments or footprints, said method comprising the steps of:
(i) providing a suspension comprising monosomes;
(ii) treating said suspension with a dissociation buffer comprising reagents that dissociate monosomes to release the ribosome protected mRNA fragments or footprints; and
(iii) filtering the mixture of step (ii) through an ultrafiltration unit comprising molecular weight cut-off (MWCO) membrane under conditions to remove impurities and elute ribosome protected mRNA fragments through the column; wherein the purified ribosome protected mRNA fragments has a length of 25-35 nucleotides.

2. The method of claim 1, further comprising first purifying said suspension comprising monosomes by
(a) adding about 20-30 fold of dilution buffer comprising reagents that reduce non-specific RNA-RNA interactions and inhibit RNase activity to the suspension; and
(b) filtering through said ultrafiltration unit comprising molecular weight cut-off (MWCO) membrane wherein said monosomes that is substantially free of impurities is retained in a retentate solution.

3. The method of claim 1, wherein said reagent in dissociation buffer is EDTA in a concentration of about 1 mM.

4. The method of claim 1, wherein said reagent in dissociation buffer is puromycin in a concentration of about 2 mM or higher.

5. The method of claim 1, wherein said membrane in ultrafiltration unit has a molecular weight cut-off (MWCO) of about 50K to about 100K Daltons.

6. The method of claim 2, wherein said reagent in dilution buffer is magnesium in a concentration of about 2.5 mM to about 5 mM.

7. The method of claim 1, wherein the ribosome protected mRNA fragments or footprints are used in RNA library preparation.

8. A method for isolation and purification of ribosome protected mRNA fragments or footprints, said method comprising the steps of:
(i) loading a suspension comprising monosomes on an ultrafiltration column comprising a molecular weight cut-off (MWCO) membrane;
(ii) adding about 20-30 fold of dilution buffer comprising reagents that reduce non-specific RNA-RNA interactions and inhibit RNase activity;
(iii) filtering through the ultrafiltration column under conditions to remove impurities and obtain monosomes that is substantially free of impurities in a retentate solution;
(iv) treating said retentate solution with a dissociation buffer comprising reagents that dissociate monosomes to release the ribosome protected mRNA fragments or footprints; and
(v) filtering the mixture of step (iv) through said ultrafiltration column under conditions to remove impurities and elute ribosome protected mRNA fragments through the column; wherein said membrane in ultrafiltration unit has a molecular weight cut-off (MWCO) of about 50K to about 100K Daltons and wherein said ribosome protected mRNA fragments has a length of 25-35 nucleotides.

9. The method of claim 8, wherein said reagent in dilution buffer is magnesium in a concentration of about 2.5 mM to about 5 mM.

10. The method of claim 8, wherein said reagent in dissociation buffer is EDTA in a concentration of about 1 mM.

11. The method of claim 8, wherein said reagent in dissociation buffer is puromycin in a concentration of about 2 mM or higher.

12. The method of claim 8, wherein the ribosome protected mRNA fragments or footprints are used in RNA library preparation.

13. A method for isolation and purification of ribosome protected mRNA fragments or footprints from a total cell lysate, said method comprising the steps of:
(i) loading said total cell lysate on an ultrafiltration column comprising a molecular weight cut-off (MWCO) membrane;
(ii) treating with a medium comprising nucleases;
(iii) adding about 20-30 fold of dilution buffer comprising reagents that reduce non-specific RNA-RNA interactions and inhibit RNase activity;
(iv) filtering through the ultrafiltration column under conditions to remove impurities and obtain monosomes that is substantially free of impurities in a retentate solution;
(v) treating said retentate solution with a dissociation buffer comprising reagents that dissociate monosomes to release the ribosome protected mRNA fragments or footprints; and
(vi) filtering the mixture of step (v) through said ultrafiltration column under conditions to remove impurities and elute ribosome protected mRNA fragments through the column; wherein said total cell lysate comprises polysomes; wherein said membrane in ultrafiltration unit has a molecular weight cut-off (MWCO) of about 50K to about 100K Daltons, and wherein said ribosome protected mRNA fragments has a length of 25-35 nucleotides.

14. The method of claim 13, wherein said nucleases are DNAse-1 and RNAse-1.

15. The method of claim 13, wherein said reagent in dilution buffer is magnesium in a concentration of about 2.5 mM to about 5 mM.

16. The method of claim 13, wherein said reagent in dissociation buffer is EDTA in a concentration of about 1 mM.

17. The method of claim 13, wherein said reagent in dissociation buffer is puromycin in a concentration of about 2 mM or higher.

18. The method of claim 13, wherein the ribosome protected mRNA fragments or footprints are used in RNA library preparation.

19. A simple and efficient method for small RNA library preparation comprising:
(i) obtaining purified ribosome protected mRNA fragments or footprints of claim 13; and
(ii) treating said ribosome protected mRNA fragments with a 5′ and 3′ end repair buffer to promote 5′ phosphorylation and 3′ dephosphorylation.

20. The method of claim 19, wherein said 5′ and 3′ end repair buffer comprising T4 Polynucleotide Kinase (T4 PNK).
